(19) United States
(12) Patent Application Publication
(10) Pub. No.: US 2008/0281086 A1
(43) Pub. Date: Nov. 13, 2008

(54) Methods, processes and materials for dispensing and recovering supported fluorous reaction components

(75) Inventors: John Andrew Gladysz, Erlangen (DE); Long V. Dinh, Piscataway, NJ (US); Dennis P. Curran, Pittsburgh, PA (US)

Correspondence Address: K&L GATES LLP, 535 SMITHFIELD STREET, PITTSBURGH, PA, 15222, US

(73) Assignee: Fluorous Technologies Incorporated

(21) Appl. No.: 12/082,763

(22) Filed: Apr. 14, 2008

Related U.S. Application Data
(62) Division of application No. 11/264,417, filed on Nov. 1, 2005.
(60) Provisional application No. 60/624,403, filed on Nov. 2, 2004.

(51) Int. Cl. A61K 51/04; C07F 5/00

(52) U.S. Cl. 534/10; 534/11; 534/15

(57) ABSTRACT

A fluorous delivery or recovery material comprising a fluorous support material having a coating thereon, the coating comprising an amount of a fluorous reaction component that may be dispensed using non-gravimetric methods is disclosed. Also disclosed are methods for dispensing a fluorous reaction component comprising dispensing by non-gravimetric methods a predetermined amount of the fluorous reaction component as a coating on a fluorous support material.

### CROSS REFERENCE TO RELATED APPLICATIONS

The present application claims priority to U.S. Provisional Application Serial No. 60/624,403 filed Nov. 2, 2004, the disclosure of which is incorporated in its entirety by reference.

### FIELD OF THE INVENTION

The present invention describes a general method for the preparation of easily delivered and recovered fluorous supported reaction components.

### BACKGROUND OF THE INVENTION

It is generally the case that organic compounds must be synthesized as pure substances through well-planned reactions and scrupulous separation/purification. In fields such as drug discovery, commodity chemical synthesis, polymer chemistry, and materials research, many types of catalysts are used, enabling different types of product selectivities. Often tens of thousands of compounds or conditions must be screened to discover the best or most active pharmaceuticals, polymerization parameters, or selectivities.

Recently, fluorous synthetic and separation techniques have attracted the interest of organic chemists. In fluorous synthetic techniques, reaction components are typically attached to fluorous groups or tags such as perfluoroalkyl groups to facilitate the separation of products. Organic compounds are readily rendered fluorous by attachment of an appropriately fluorinated phase label or tag. In general, fluorous-tagged molecules partition preferentially into a fluorous phase. This fluorous phase is typically insoluble in or immiscible with organic or inorganic solvents under standard reaction conditions. This characteristic of fluorous compounds has lead to the development of fluorous biphasic catalysis, such as liquid/liquid fluorous biphasic catalysis (I. T. Horvath and J. Rabai, Science, 1994, 266, 72). Fluorous biphasic catalysis provides a simple solution to the product/reagent or product/catalyst separation problems inherent in chemical systems. By utilizing a fluorous reagent or catalyst, separation of the fluorous reaction components from the organic reaction components is accomplished via a fluorous phase/organic phase liquid/liquid or liquid/solid separation protocol wherein the fluorous reagent or catalyst selectively partitions into the fluorous phase and the organic products partition into the organic phase. Several fluorous reaction and separation techniques are disclosed, for example, in U.S. Pat. Nos. 6,156,896; 5,859,247 and 5,777,121. In addition, several fluorous reaction and separation techniques are disclosed in U.S. patent application Ser. Nos. 09/506,779; 09/565,087; 09/583,247; 09/932,903; 09/977,944 and 10/094,345.

Catalyst delivery is an important issue in all catalytic processes. The precise amount of catalyst is an important variable. In most cases, the desired amount is weighed out on a laboratory balance or scale (gravimetric delivery). However, this can be subject to error, especially when the catalyst is very active and the required amounts very small. Furthermore, gravimetric delivery can be inconvenient and time consuming when multiple reactions are conducting in serial or in parallel. For these and other reasons, many catalysts are sold on supports, such as amorphous carbon, silica gel, or polymer beads. These delivery issues are not restricted to catalysts and extend to other reaction components (such as, for example, reagents, reactants, and scavengers) as well.

Recent reports demonstrate some advances regarding the preparation of fluorous catalysts on supports. Bannwarth, et al. have reported the coating of fluorous palladium complex onto fluorous silica gel (Tzschucke, C. C.; Markert, C.; Glatz, H.; Bannwarth, W. Angew. Chem., Int Ed. 2002, 41, 4500; Angew. Chem. 2002, 114, 4678). These were applied to Suzuki and Sonogashira coupling reactions. Biffis, et al. have reported the coating of a fluorous dirhodium complexes onto fluorous silica gel (Biffis, A.; Zecca, M.; Basato, M. Green Chemistry, 2003, 5, 170). This system catalyzes the silylation of alcohols by trialkylsilanes. However, these methods have limited application in that the silica gel is a powder and the catalyst must be delivered gravimetrically (using a costly analytical balance or scale). It is also impossible to fabricate fluorous silica gel into easily retrieved objects like tapes, meshes, or rods.

It would therefore be desirable to develop supported fluorous catalysts and other reaction components that can be easily synthesized and delivered and retrieved by more convenient or alternative means.

### SUMMARY

The present invention addresses one or more of the above-mentioned needs by providing a fluorous delivery or recovery material comprising a fluorous support material having a coating thereon, the coating comprising an amount of a fluorous reaction compound or fluorous reaction component, wherein a non-gravimetric method is used to deliver the fluorous support material having a desired amount of the fluorous reaction component.

In another embodiment, the present invention provides a method for dispensing a fluorous component comprising dispensing by a non-gravimetric method a desired amount of the fluorous reaction component, wherein the fluorous reaction component is a coating on at least a fluorous support material. The method further comprises adding the fluorous support material coated with the desired amount of the fluorous reaction component to a reaction vessel.

According to another embodiment, the present invention provides a method for forming a fluorous delivery and recovery material. The method comprises depositing a fluorous reaction component as a coating on at least a portion of a surface of a fluorous support material, wherein the fluorous support material is capable of being applied or dispensed to a reaction by a non-gravimetric method.

It should be understood that this invention is not limited to the embodiments disclosed in this summary, but it is intended to cover modifications that are within the spirit and scope of the invention, as defined by the appended claims.

### DESCRIPTION OF FIGURES

FIG. 1 illustrates one embodiment of a method of conducting a reaction using a fluorous reaction component coated on a fluorous support material.

FIG. 2 illustrates an embodiment of the method of conducting a reaction using a fluorous reaction component wherein the reaction component precipitates on the fluorous support material.

### DETAILED DESCRIPTION AND DISCUSSION

Other than in the operating examples, or where otherwise indicated, all numbers expressing quantities of ingredients, reaction conditions and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

All patents and publications set forth herein are incorporated herein by reference. Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

The present invention describes a new delivery system for fluorous reaction components supported on fluorous support materials. In one embodiment of the current invention, a method for dispensing a fluorous compound or material is described, the method comprising the steps of: a) preparing a fluorous support material coated with the fluorous reaction component, such as, for example, a fluorous compound, fluorous oligomer or fluorous polymer; and b) dispensing a predetermined amount of the fluorous reaction component, such as the fluorous compound, fluorous oligomer or fluorous polymer by a non-gravimetric method, such as, by cutting a unit length or cutting a unit area of the coated fluorous support material. The coated fluorous support material containing the fluorous compound can be an insoluble fluorous oligomer, fluorous polymer or fluorous bonded phase that is dispensed into a chemical reaction medium. In some embodiments, the chemical reaction medium is treated under conditions to generate at least one new chemical product, and further the coated fluorous support material is sometimes separated from the chemical reaction medium. According to various embodiments, the use of a fluorous support material for the delivery and recovery of a fluorous reaction component, as described herein, is more convenient and better than use of other support materials for delivery and recovery of reaction components.

As used herein, the term “non-gravimetric method” means a method for measuring an amount of a material other than by measuring the weight of the material. Non-limiting examples of non-gravimetric methods of measuring include measuring by unit length, measuring by unit area, measuring by volume, or measuring by counting out a number of pieces. According to certain embodiments, gravimetric methods, such as weighing, are not necessary for delivering know or predetermined quantities of a fluorous reaction component, such as, for example a fluorous reagent or fluorous catalyst. As used herein, the terms “coated”, “deposited”, “adsorbed”, and “absorbed,” when used to describe the fluorous reaction component on at least a portion of a surface of the fluorous support material, means the fluorous reaction component forms at least a partial layer on at least a portion of a surface of the fluorous support material.

The fluorous compound or fluorous reaction component can be any type of fluorous reaction component, including but not limited to, catalyst, reagent, reactant, scavenger, substrate, product, such as, for example, those fluorous compounds set forth herein and in co-pending U.S. application Ser. Nos. 10/617,431 and 10/664,105, which are incorporated by reference herein in their entirety. When the fluorous material is a catalyst, it can be recovered and reused after the reaction. In some embodiments, the fluorous component is transformed in the reaction. In other embodiments, at least one organic compound is added to the reaction mixture, and this is transformed.

According to certain embodiments, the fluorous reaction component coated on the fluorous support material may exhibit temperature dependent solubility, as set forth in U.S. application Ser. No. 10/664,105. As used herein, the term “temperature dependent solubility” means that for a given solvent, at a first temperature, the fluorous reaction component is substantially insoluble in the solvent. Thus, according to certain embodiments, the fluorous reaction component may remain substantially adsorbed or deposited on at least a surface of the fluorous support material. According to another embodiment, the fluorous reaction component may remain as a solid precipitate in the reaction medium. When the temperature of the reaction medium is changed to a second temperature, the solubility of the fluorous reaction component in the solvent increases and the fluorous reaction component becomes at least partially dissolved in the solvent. When the temperature of the reaction medium is changed to a third temperature, the fluorous reaction component becomes substantially insoluble in the solvent and deposits on at least a portion of the surface of the fluorous support material.

In another embodiment, a method for dispensing a fluorous compound or material is described, the method comprising the steps of: a) preparing a fluorous support material coated with a fluorous reaction component, such as, a fluorous compound, fluorous oligomer or fluorous polymer; and b) dispensing a predetermined amount of the fluorous compound, fluorous oligomer or fluorous polymer by a non-gravimetric method, such as, counting units of the coated fluorous support material. Reaction and separation features are generally as described above.

A highly fluorinated (fluorous) reaction component (for example, a catalyst) may be coated onto a fluorous support material. As used herein, the term “support material” is meant to include any fluorous receptive surface, such as, for example Teflon® (Teflon® is a registered trademark of DuPont for polytetrafluoroethylene) tape, other fluoropolymer tape, or other type of fluoropolymer surface or object (sheets, meshes, rods, bars, etc.). As use herein, the term “coating” is meant to include at least a portion of at least one layer containing a fluorous material, such as a fluorous reaction component, deposited over, but not necessarily adjacent to, the support material, wherein the concentration of the fluorous reaction component per unit length or unit area is known. The desired fluorous reaction component quantity may then be delivered by length or area of the coated material, avoiding the need for gravimetric methods involving specialized equipment such as an analytical balance. Alternatively, the object can be coated with the exact catalyst charge needed for a given application. Or the coated objects with a known coated level can be dispensed by counting, such as, for example, described below.

In one embodiment for preparing supported fluorous reaction components, a known amount of the fluorous compound is dissolved in a suitable solvent at room temperature or above. A known length of fluorous support material, for example, Teflon® tape is then added, and the solvent removed by evaporation, distillation, a gas stream, or a similar technique. The fluorous reaction component may be deposited as a coating on at least a portion of at least one surface of the fluorous support material. The fluorous reaction component can also be coated onto the tape by cooling, or the addition of a second solvent, or the addition of another agent or other means that reduce the solubility of the fluorous material, thereby depositing the fluorous reaction component as a coating on at least a portion of at least a surface of the fluorous support material. Other types of fluoropolymers may be used, as well as other fluoropolymer morphologies (sheets, meshes, rolls, rods, beads etc.). The catalyst adsorbs onto the tape or similar material. With colored catalysts, this is evidenced by a change in the color of the tape. For some applications, including dispensing by counting or volume, fluorous silica gel and related fluorous bonded phase materials can also be used as fluorous support materials.

The absorption may be uniform, and the amount of catalyst deposited on a given length or area may be easily calculated from the amount originally dissolved in solution and the total length or area of the original fluorous support material. Thus, gravimetric methods involving an expensive analytical balance or scale are no longer needed for catalyst delivery, representing a significant advance over prior art. For example, the person performing the delivery can cut the tape to length corresponding to the desired loading for the reaction. Fluorous reaction components, for example, very small catalyst amounts, can be easily and conveniently delivered by non-gravimetric methods (i.e., without weighing out the amount of fluorous reaction component). After the catalytic reaction, the tape or other fluorous support material can be easily separated from the products, for example, by filtration, decantation, or simply by removing or “fishing out” the fluorous support material coated with the fluorous reaction component from the reaction medium.

Fluoropolymers and similar materials fabricated in the form of reaction vessels or reaction vessel components (stir blades, stir bars, plugs, interior liners) may also be employed as fluorous support materials. Catalyst coating may be affected in a similar fashion as described herein above. In these cases, the amount of catalyst absorbed corresponds preferably but not exclusively to the initial charge desired for a given reaction or application.

In the case where more catalyst is needed than is supported on a single fluorous support object, the catalyst can be dispensed in the needed quantity simply by counting out a number of the objects corresponding to the desired amount of catalyst. Exact counting can be done by hand or machine, but estimated counting suffices for many applications. For example, the count of a requisite number of fluorous support beads or objects can be estimated simply by pouring the beads into a container of a suitable size or volume, and measuring the beads or objects against the volume markings on the container (for example, a measuring cup) and then pouring the measured quantity of beads out into the reaction vessel.

In some embodiments of this invention, the fluorous reaction component may have a general formula: D[(R)ₙ(Rf)ₘ]_{y} wherein D has a structure selected from the group consisting of an organic group, P, OH, OR, N, S, As, and Si, R is independently, the same or different, a hydrocarbon moiety, Rf is independently, the same or different, a fluorous moiety, n is an integer equal to at least 0, m is an integer greater than 0, and y is an integer between 1 and the maximum number of bonding attachments of D.

In other embodiments of this invention, the fluorous reaction component may have a general formula: Mₓ{L[(R)ₙ(Rf)ₘ]_{y}}_{z} wherein M is a metal selected from the group consisting of a transition metal, a lanthanide metal, thorium, uranium, and main-group metals, L is a ligand core having a structure selected from the group consisting of C, N, O, P, As, S and Si, R is independently, the same or different, a hydrocarbon moiety, Rf is independently, the same or different, a fluorous moiety, n is an integer equal to at least 0, m is an integer greater than 0, y is an integer between 1 and the maximum number of bonding attachments of L, z is an integer between 1 and the maximum number of ligands attachable to M, and x is an integer from 1 to 4. In one embodiment, the fluorous compound or catalyst may have the formula

ClRh[P((CH₂)ₘ(CF₂)ₙCF₃)₃]₃ (where m=1-8, n=5-13).

As used herein the terms “fluorinated hydrocarbon” and “fluorohydrocarbon” include organic compounds or substituents in which at least one hydrogen atom bonded to a carbon atom is not replaced with a fluorine atom. The term “perfluorocarbon” means an organic compound or substituent in which all hydrogen atoms bonded to carbon atoms are replaced with fluorine atoms. Perfluorocarbon substituents may have the general formula CₙF₂ₙ₊₁, where n is an integer greater than or equal to 1. The term “fluorous compound” (for example, a fluorous reaction component) is defined as an organic molecule, a portion or domain of which is rich in carbon-fluorine bonds (for example, fluorocarbons or perfluorocarbons, fluorohydrocarbons, fluorinated ethers, fluorinated amines and fluorinated adamantyl groups). For example, perfluorinated ether groups can have the general formula —[(CF₂)ₓO(CF₂)_{y}]_{z}CF₃, wherein x, y and z are integers. Perfluorinated amine groups can, for example, have the general formula —[(CF₂)ₓ(NR^{a})CF₂)_{y}]_{z}CF₃, wherein R^{a} can, for example, be (CF₂)ₙCF₃, wherein n is an integer. Fluorous alkyl groups, fluorous ether groups and fluorous amine groups suitable for use in the present invention need not be perfluorinated, however. Typically this means that the “fluorous” organic molecule must contain a significant number of fluorine atoms. About 20 wt % fluorine to less than about 80 wt % of the total composition is desirable for fluorous reaction components (for example, fluorous catalysts). Typically, at least 50 wt % fluorine relative to total composition of fluorous molecule or material is desirable. A few examples of suitable fluorous groups, Rf, for use in the present invention include, but are not limited to, —C₄F₉, —C₆F₁₃, —C₈F₁₇, —C₁₀F₂₁, —C(CF₃)₂C₃F₇, —C₄F₈CF(CF₃)₂, —CF₂CF₂OCF₂CF₂OCF₃, —CF₂CF₂(NCF₂CF₃)CF₂CF₂CF₃, —C₆F₁₂H, —C₈F₁₆H, fluorous adamantyl groups, and/or mixtures thereof.

Perfluoroalkyl groups and hydrofluoroalkyl groups are well suited for use in the catalysts applied in the present invention. For example, Rf can be a linear perfluoroalkyl group of 3 to 20 carbons, a branched perfluoroalkyl group of 3 to 20 carbons, and a hydrofluoroalkyl group of 3 to 20 carbons. Hydrofluoroalkyl groups may typically include up to one hydrogen atom for each two fluorine atoms.

Certain organic-based fluorous reaction components, such as, for example, fluorous catalysts, may have the formula: D[(R)ₙ(Rf)ₘ]_{y} wherein D is an organic or heteroatom core to which at least one fluorous moiety is bonded, i.e. [(R)ₙ(Rf)ₘ], which may include the hydrocarbon domain, (R)ₙ, and the fluorous domain, (Rf)ₘ. For metal-based fluorous reaction components the formula may be: Mₓ{L[(R)ₙ(Rf)ₘ]_{y}}_{z} wherein M is a metal selected from the group consisting of a transition metal, a lanthanide metal, thorium, uranium, and main-group metals, L is a ligand core having a structure selected from the group consisting of C, N, O, P, As, S and Si, R is independently, the same or different, a hydrocarbon moiety, Rf is independently, the same or different, a fluorous moiety, n is an integer equal to at least 0, m is an integer greater than 0, y is an integer between 1 and the maximum number of bonding attachments of L, z is an integer between 1 and the maximum number of ligands attachable to M, and x is an integer from 1 to 4.

In both the above formulas, (Rf)ₘ is a fluorous domain, (R)ₙ is a hydrocarbon domain that may contain H and C, or may contain groups containing O, N, S, P, As and Si in addition to H and C in the backbone and/or as substituents, but wherein (R)ₙ is hydrogen atom rich in comparison to (Rf)ₘ, and wherein n is an integer equal to at least zero or any whole number, preferably 0, 1, 2; and wherein m is any whole number; and wherein L is a ligand core containing C, N, O, P, As, S, Si and, in combination with the foregoing, H; and wherein y is the maximum number of fluorous moieties attachable to L or to D, as the case may be; and wherein z is the maximum number of ligands attachable to the metal M. Changing the ratio between n and m could have major impact on the reactivity of a fluorous reaction component, such as fluorous catalyst, because fluorous domains are strongly electron withdrawing. Addition of hydrocarbon domains (at least about 2, preferably at least 3 “—CH₂—” or similar groups, for example) as spacer groups between L or D and the fluorous domain generally reduces the electron withdrawing effect of the fluorous domain on M or D of the fluorous reaction components. The fluorous reaction components typically may contain a plurality of such fluorous moieties (i.e. y is greater than 1) having a significant proportion of fluorine atoms. By significant proportion is meant at least about 20 wt %, typically about 20 to 80 wt %, and in some embodiments from about 50 to 80 wt % of fluorine to total weight of the composition. Variability within (R)ₙ, (Rf)ₘ and M or D may be introduced to accommodate components, such as, catalysts having, for example, multiple metal centers, or variation in the types of ligands. Thus, when the particular subscript n, m, y, or z is greater than 1 each n, m, y and z may be the same or different.

The fluorous domain, (Rf)ₘ, typically may have a rod-like molecular structure especially when derived from longer straight chain carbon containing backbones. In addition to L, the fluorous reaction component, such as fluorous catalyst, may contain other ligands. Typically, other ligands known in the art to be used in homogeneous reactions, such as catalysis, for a particular reaction may be incorporated into the fluorous reaction components when the fluorous reaction component is a modification or derivative of a known parent non-fluorous reaction component. Variability within (R)ₙ, (Rf)ₘ and M or D may be introduced to accommodate systems having, for example, multiple metal centers, or variation in the types of ligands. Such systems are well known homogeneous fluorous reaction components, such as catalysts, and are amendable to fluorofunctionalization (“ponytailing”) as described herein.

Certain non-limiting examples of fluorous reaction components having the formula D[(R)ₙ(Rf)ₘ]_{y} or Mₓ{L[(R)ₙ(Rf)ₘ]_{y}}_{z} are described herein. Thus, for example, for the catalyst Cl—Rh—{P[CH₂—CH₂(CF₂)₆F]₃}₃ (the non-fluorous parent compound of which is known as Wilkinson's catalyst, and is used for hydrogenation reactions), Rh corresponds to the Mₓ wherein M=Rh, x=1; P corresponds to L; —CH₂—CH₂— corresponds to (R), n=1; —(CF₂)₆—F to (Rf), m=1; the subscript 3 to y and the final subscript 3 to z. Similarly, for the fluorous reagent, CH₂═P[CH₂—CH₂(CF₂)₇CF₃)₃, (the non-fluorous parent compound of which is known as a Wittig reagent), D in the above formula is CH₂═P; —CH₂CH₂— is (R); n=1; —(CF₂)₇CF₃ is (Rf); m=1; and y is 3. Another fluorous phosphine reagent or catalyst has the formula P[CH₂—CH₂(CF₂)₇CF₃)₃, where D in the above formula is P; —CH₂CH₂— is (R); n=1; —(CF₂)₇CF₃ is (Rf); m=1; and y is 3. A fluorous Brønsted acid catalyst may be HOOC—[CH₂—CH₂(CF₂)₇CF₃]. Thus, HOOC-group corresponds to D in the formula. A fluorous palladium catalyst suitable for reactions, such as, Suzuki type coupling reactions, may have the formula [(CF₃(CF₂)₇CH₂CH₂)₂S]₂PdCl₂, where Pd corresponds to the Mₓ wherein M=Pd, x=1; S corresponds to L; —CH₂—CH₂— corresponds to (R), n=1; —(CF₂)₇CF₃ to (Rf), m=1; the subscript 2 to y and the final subscript 2 to z. A fluorous dirhodium tetracarboxylate catalyst may have the formula Rh₂(O₂CR)₄ {R=the meta-disubstituted phenyl group C₆H₃—3,5—((CF₂)₅CF₃)₂}, where Rh corresponds to the Mₓ wherein M=Rh, x=2; O₂C corresponds to L; C₆H₃ corresponds to (R), n=1; —(CF₂)₅CF₃ to (Rf), m=2; the subscript 1 to y and the final subscript 4 to z. One skilled in the art will recognize that other fluorous reaction components having the general formulae, as set forth above, can also be used in the various methods and compositions set forth in the present disclosure without departing from the invention as set forth and claimed herein.

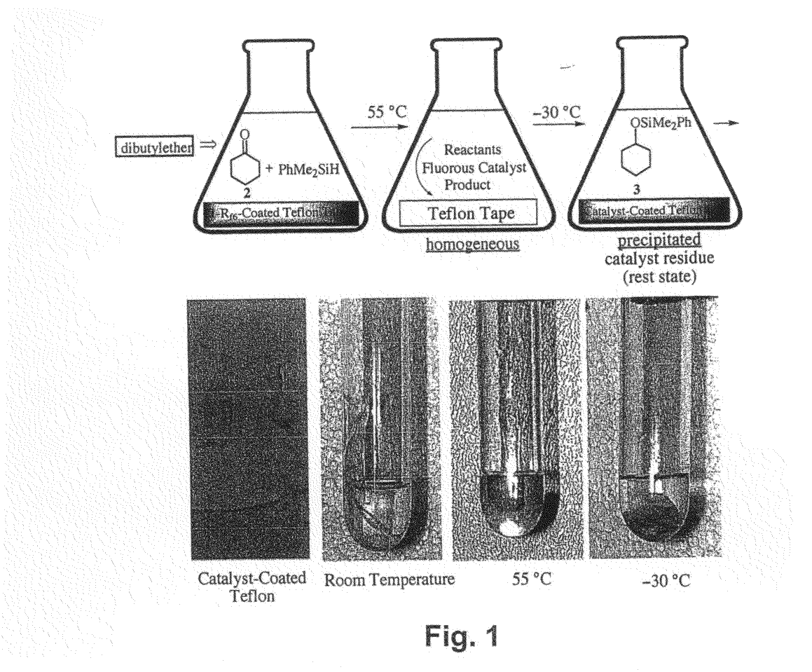

One example of the method for conducting a reaction utilizing the compositions of the present disclosure may comprise the following steps which are illustrated in FIG. 1. A requisite amount of the fluorous reaction component (denoted as 1-R_{f6}) coated on a measured amount of the fluorous support material, such as, for example, a measured length of Teflon® tape or a known quantity of fluoropolymer beads, may be added to a reaction vessel along with a solvent and at least one organic reactant. In certain embodiments, the solvent may be chosen such that the fluorous reaction component exhibits temperature dependent solubility in the solvent. That is, at a first reaction temperature, the fluorous reaction component remains substantially deposited as a coating on the fluorous support material. As the reaction medium temperature is changed, such as heated, to a second temperature, wherein the fluorous reaction component has increased solubility in the solvent, the fluorous reaction component becomes at least partially dissolved in the solvent. After a certain amount of time the reaction temperature may be changed, such as cooled, from the second temperature to a third temperature, wherein the solubility of the fluorous reaction component in the solvent is reduced and a significant portion of, and in some embodiments, substantially all of the fluorous reaction component re-deposits on at least a portion of the surface of the fluorous support material. The fluorous reaction component coated on the fluorous support material may be removed from the reaction medium, such as, for example, by filtration, decantation, removal of the solvent, or “fishing out” of the fluorous support material. In certain embodiments, the fluorous reaction component coated on the fluorous support material may be used in at least one subsequent reaction.

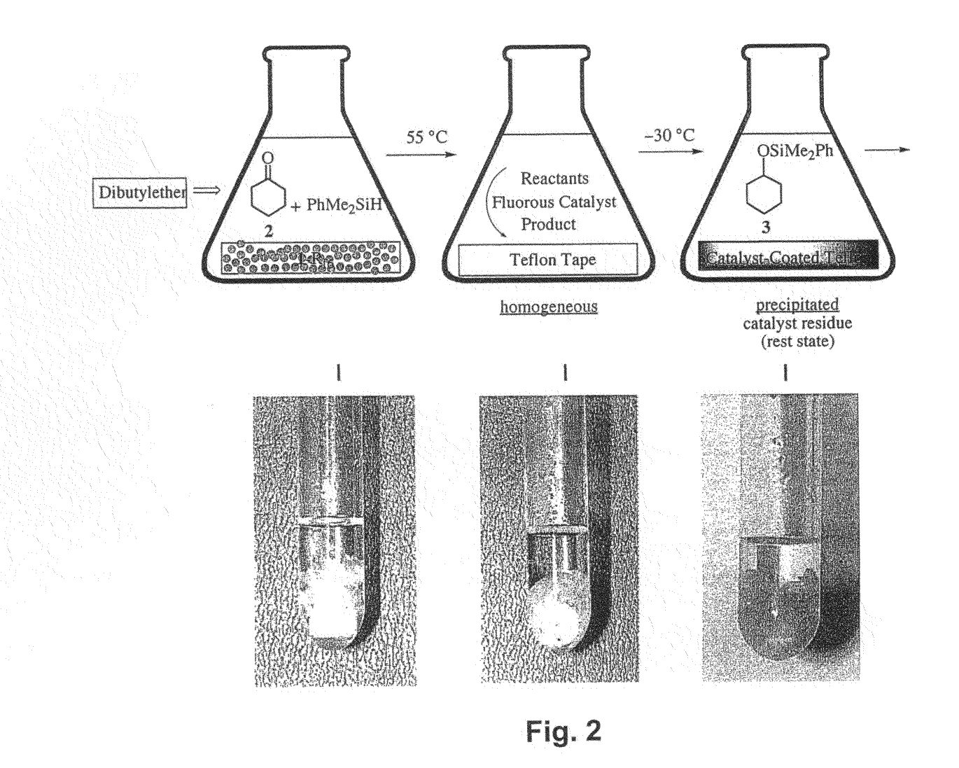

Another method for conducting a reaction utilizing the compositions of the present disclosure may comprise the following steps as illustrated in FIG. 2. According to this embodiment, the fluorous support material may be utilized to aid in separation of a fluorous reaction component from the reaction medium. For example, the fluorous reaction component (1-R_{f6}), at least one organic reactant, and the uncoated fluorous support material are added to the reaction vessel. A solvent is chosen such that the fluorous reaction component exhibits temperature controlled solubility in the solvent. At a first temperature, the fluorous reaction component is substantially insoluble in the solvent. As the reaction medium temperature is changed, such as heated, to a second temperature, wherein the fluorous reaction component exhibits a higher solubility in the solvent, the fluorous reaction component becomes at least partially dissolved in the solvent. After a certain amount of time the reaction temperature may be changed, such as cooled, from the second temperature to a third temperature, wherein the solubility of the fluorous reaction component in the solvent is reduced and a significant portion of, and in some embodiments, substantially all of the fluorous reaction component deposits on at least a portion of the surface of the fluorous support material. The fluorous reaction component coated on the fluorous support material may be removed from the reaction medium, such as by a method as described above. In certain embodiments, the fluorous reaction component coated on the fluorous support material may be used in at least one subsequent reaction.

In certain embodiments of the methods for conducting a reaction, the fluorous reaction component may react with the at least one organic reactant while dissolved in the solvent, although in other embodiments, the fluorous reaction component may react with the at least one organic reactants while coated on the fluorous support material.

The present invention will be described further by reference to the following examples. The following examples are merely illustrative of the invention and are not intended to be limiting. Unless otherwise indicated, all parts are by weight.

### EXAMPLES

### Example 1

### Fluorous Rhodium Catalyst Adsorbed on Teflon® Tape (FIG. 1)

The fluorous rhodium catalyst ClRh[P((CH₂)₂(CF₂)₅CF₃)₃]₃ (0.013 g, 0.0039 mmol) was added to a 10 mL round-bottom flask. Then 11.0 mL of CF₃C₆F₁₁ was then added to dissolve the catalyst, giving a yellow solution. Teflon® tape (2 strips of 5.0 cm length and 0.0075 mm thickness; in unfolded form) was added to the catalyst solution. Solvent was then allowed to evaporate under a nitrogen or argon stream. The Teflon® tape became coated, with a yellowish color.

### Example 2

### Application of Fluorous Rhodium Catalyst: Hydrosilylation of Cyclohexanone

This procedure is representative only, as many variations can be conducted. A 10 mL screw-top vial was charged with Teflon® tape (2 pieces of 5.0 cm length and 0.0075 mm thickness.) that had been coated with ClRh[P((CH₂)₂(CF₂)₅CF₃)₃]₃ (corresponding to 0.0013 g, 0.0039 mmol of catalyst), tridecane GC standard (0.2001 g, 1.085 mmol added gravimetrically), cyclohexanone (0.2610 g 2.650 mmol), dimethylphenylsilane (0.4301 g, 3.180 mmol) and dibutylether (5.0 mL). The sample was stirred at 55° C. for 3 hours. An aliquot of 10.0 μL was taken and diluted with 1.0 mL of dibutylether. The sample was analyzed by GC. The reaction vessel was stored at −30° C. for 4 hours. The clear organic (dibutylether) phase was carefully removed from the supported catalyst via syringe. The residue was extracted 2 times with cold dibutylether (0.5 mL, −30° C.). The vial with the supported catalyst was again charged with tridecane, cyclohexanone, dimethylphenylsilane, and dibutylether, and the procedure repeated (yields for cyclohexyl dimethylphenylsilyl ether for 3 cycles: 94%, 88%, 81%).

### Example 3

### Application of Fluorous Rhodium Catalyst: Hydroboration of Norbornene

This procedure is representative only, as many variations can be conducted. A flask was charged with norbornene (0.0746 g, 0.792 mmol), catecholborane (0.100 g, 0.834 mmol), and Teflon® tape (2 strips of 5.0 cm length and 0.0075 mm thickness) that had been coated with ClRh[P((CH₂)₂(CF₂)₅CF₃)₃]₃ (corresponding to 8.95×10⁻⁴ mmol of catalyst). Dibutylether (4.0 mL) was added. The mixture was kept in a 55° C. bath (3 hours), then cooled to −30° C. (4 hours). The clear organic phase was carefully removed from the supported catalyst via syringe. The residue was extracted twice with cold dibutylether (0.5 mL, −30° C.). The combined dibutylether extract was combined with ethanol/THF (10 mL, 1:1 v/v) and NaOH (5 mL, 2 M in H₂O). The mixture was placed in an ice bath and 30% H₂O₂ (1.0 mL, 8.8 mmol) was added dropwise with stirring. After 0.5 h, the ice bath was removed. After 6 hours, the mixture was extracted with ether (3×15 mL). The extract was washed with NaOH (10 mL, 0.5 M in H₂O), H₂O (25 mL), and brine (15 mL), and dried over MgSO₄. Solvent was removed by vacuum to give exo-norborneol as a white solid (0.2633 g, 2.35 mmol, 90%). The vial with the supported catalyst was again charged with norbornene, catecholborane, dibutylether and the procedure repeated (yields of exo-norborneol for 3 cycles: 90%, 88%, 85%).

### Example 4

### Fluorous Phosphine Catalyst Adsorbed on Teflon® Tape

A 10 mL round-bottom flask was charged with the fluorous phosphine P((CH₂)₂(CF₂)₇CF₃)₃ (0.0686 g, 0.050 mmol), and Teflon® tape (3 strips of 5.0 cm length and 0.0075 mm thickness; in unfolded form). n-Octane (2.0 mL) was then added. The mixture was heated to 65° C. and the fluorous phosphine dissolved. The solution was cooled to 0° C. for 4 hours, and the catalyst precipitated onto the tape. The solvent was then removed carefully by syringe. Alternatively, the solvent can be removed under vacuum at room temperature. The remaining catalyst-coated Teflon® tape was allowed to dry under an argon or nitrogen stream.

### Example 5

### Application of Fluorous Phosphine Catalyst: Hydroalkoxylation of Methyl Propiolate

This procedure is representative only, as many variations can be conducted. A 10 mL screw-top vial was charged with catalyst-coated Teflon® tape (3 strips of 5.0 cm length and 0.0075 mm thickness; corresponding to 0.0686 g, 0.0500 mmol of total catalyst P((CH₂)₂(CF₂)₇CF₃)₃), n-undecane GC standard (0.3-0.5 mmol added gravimetrically), benzylic alcohol (0.1082 g, 1.000 mmol), methyl propiolate (0.0421 g, 0.500 mmol) and n-octane (2.0 mL). The sample was stirred at 65° C. for 8 hours, and stored at −30° C. overnight. The light yellow organic phase was carefully removed from the supported catalyst via syringe. The residue was shaken with cold n-octane (0.8 mL, −30° C.), and the octane layer similarly separated. The organic phases were combined. An aliquot (0.200 mL) was filtered through a silica gel plug (1 cm) with ethyl acetate/hexanes (10 mL, 1:10 v/v). The filtrate was analyzed by GC (0.0010 mL autoinjection). The vial with the supported catalyst was again charged with n-undecane, benzylic alcohol, methyl propiolate, and octane, and the procedure repeated (yields of E-C₆H₅CH₂OCH═CHCO₂CH₃for six cycles: 82%, 82%, 81%, 83%, 81%, 82%).

### Example 6

### Fluorous Palladium Catalyst Adsorbed on Teflon® Tape

The fluorous palladium complex [(CF₃(CF₂)₇CH₂CH₂)₂S]₂PdCl₂ (0.0041 g, 0.0020 mmol) and CF₃C₆F₅ (1.0 mL) was added into a 10 mL round-bottom flask. All catalyst dissolved to give a yellow solution. Teflon® tape (2 strips of 5.0 cm length and 0.0075 mm thickness; in unfolded form) was added to the catalyst solution. Solvent was then allowed to evaporate under a nitrogen or argon stream. The Teflon® tape became coated, with a yellowish color.

### Example 7

### Application of Fluorous Palladium Catalyst: Suzuki Coupling of p-Bromotoluene and PhB(OH)₂

This procedure is representative only, as many variations can be conducted. A tube was charged with Teflon® tape (2 strips of 5.0 cm length and 0.0075 mm thickness; in unfolded form) that had been pre-coated with the catalyst [(CF₃(CF₂)₇CH₂CH₂)₂S]₂PdCl₂ (corresponding to 0.0041 g, 0.0020 mmol). A stock DMF solution (2.00 mL) that was 0.50 M in p-bromotoluene (1.00 mmol) and 0.75 M in PhB(OH)₂ (1.50 mmol) was added, immediately followed by aqueous K₃PO₄ (1.33 M; 1.50 mL, 2.00 mmol). Reactions were conducted at 50° C. for 5 hours. The reaction was cooled to −30° C. The DMF layer was carefully removed via syringe, and the residue extracted once more with cold DMF (−30° C., 1.00 mL). The combined DMF extracts were analyzed by GC with dibutylether as an internal standard. The tube with the catalyst support was recharged with the DMF solution of p-bromotoluene and PhB(OH)₂, and then the aqueous solution of K₃PO₄ (1.50 mL). An identical second cycle was conducted with the remaining tape (yields p-phenyltoluene for 3 cycles: 97%, 78%, 46%).

### Example 8

### Fluorous Dirhodium Catalyst Adsorbed on Teflon® Tape

The fluorous dirhodium tetracarboxylate Rh₂(O₂CR)₄ {R=the meta-disubstituted phenyl group C₆H₃—3,5—((CF₂)₅CF₃)₂} (0.0097 g, 0.01 mmol) was added to a 10 mL round-bottom flask. Then 1.0 mL of CF₃C₆F₁₁ was then added to dissolve the catalyst, giving a greenish solution. Teflon® tape (2 strips of 5.0 cm in length and 0.0075 mm in thickness; in unfolded form) was added into the catalyst solution. Solvent was then allowed to evaporate under a nitrogen or argon stream. The Teflon® tape became coated, with a greenish color.

### Example 9

### Application of Fluorous Dirhodium Catalyst: Cyclopropanation of Styrene

This procedure is representative only, as many variations can be conducted. A 10 mL Schlenk flask was charged with Teflon® tape (2 strips of 5.0 cm length and 0.0075 mm thickness) that had been coated with the catalyst Rh₂(O₂CR)₄ {R═C₆H₃—3,5—((CF₂)₅CF₃)₂} (corresponding to 0.0097 g, 0.01 mmol). Toluene (5.0 mL) was added and the mixture was heated to 60° C. Methyl diazoacetate (0.100 g, 1.0 mmol) was added over 8 hours and then a tenfold excess of styrene (1.13 mL, 10.0 mmol) was added. The reaction was allowed to stir for an additional 5 hours, and then cooled to −30° C. for 4 hours. The toluene phase is removed by syringe and the residue washed twice with 0.5 mL of cold (−30° C.) toluene. To the combined toluene phases was added dibenzyl ether as a GC standard. Analysis by GC showed a 70% yield of cyclopropyl benzene. An identical second cycle was conducted with fresh toluene, methyl diazoacetate, styrene, and the remaining tape (yield of cyclopropyl benzene 66%).

### Example 10

### Application of Fluorous Rhodium Catalyst: Hydrosilylation of Cyclohexanone with Recovery of Catalyst on Teflon® Tape and Catalyst Recycling (FIG. 2)

A 10 mL round bottom flask inside a glovebox was charged with cyclohexanone (0.2597 g, 2.650 mmol), tridecane (0.2002 g, 1.086 mmol), PhMe₂SiH (0.4301 g, 3.156 mmol), five pieces of uncoated Teflon® tape (30×12×0.0075 mm, l×w×thickness), freshly made ClRh[P((CH₂)₂(CF₂)₅CF₃)₃]₃ (0.0130 g, 0.15 mol %, 0.0039 mmol) and dibutylether (5.0 mL). The flask was capped with a septum and heated in a bath (55° C.) with stirring. A yellow monophasic solution formed. The reaction was monitored by GC every 15 min. (0.005 mL aliquot in a GC sample vial and diluted with 1.0 mL of dibutylether). GC analysis (0.001 mL: autoinjection) indicated that the maximum yield was reached within 3 h. The reaction flask was cooled to −30° C. for 4 h. Then the dibutylether was removed by syringe and the residue containing the tape pieces was washed with cold dibutylether (2×0.50 mL). The vessel was then allowed to warm to room temperature and another batch of cyclohexanone (0.2591 g, 2.640 mmol), tridecan (0.2005 g, 1.086 mmol), and PhMe₂SiH (0.4309 g, 3.156 mmol) was added for the next cycle. The reaction was repeated and monitored by GC every 15 min as before. After completion, an identical workup procedure was followed. The substrates were reloaded and the procedure was repeated two more times (GC yields for cyclohexyl dimethylphenylsilyl ether for 4 cycles: 98%, 97%, 96%, 65%).

### Comparative Example 1

### Application of Fluorous Rhodium Catalyst Without a Fluorous Support Material: Hydrosilylation of Cyclohexanone

A 10 mL round bottom flask inside a glovebox was charged with cyclohexanone (0.2605 g, 2.654 mmol), tridecane (0.2002 g, 1.086 mmol), PhMe₂SiH (0.4301 g, 3.156 mmol), freshly made ClRh[P((CH₂)₂(CF₂)₅CF₃)₃]₃ (0.0891 g, 1.0 mol %, 0.2654 mmol, weighed out on an analytical balance) and dibutylether (5.0 mL). The flask was capped with a septum and heated in a bath (65° C.) with stirring. A yellow monophasic solution formed. After 8 hr, the reaction was stopped. Then an aliquot (0.005 mL) was removed and diluted with dibutylether. GC analysis (0.001 mL autoinjection) showed a yield of 98% (2.601 mmol). The flask was cooled to −30° C. for 4 hr. Then the dibutylether was removed by syringe and the residue was washed with cold dibutylether (2×0.50 mL). The vessele was then allowed to warm to room temperature and another batch of cyclohexanone (0.2595 g, 2.640 mmol), tridecane (0.2005 g, 1.087 mmol), and PhMe₂SiH (0.4309 g, 3.156 mmol) was added for the next cycle. After 8 hr, the reaction was stopped. Then an aliquot of 0.005 mL was removed and diluted with dibutylether. GC analysis (0.001 mL autoinjection) showed a yield of 98% (2.587 mmol). An identical workup procedure was followed. The substrates were reloaded and the procedure was repeated two more times (GC yields for cyclohexyl dimethylphenylsilyl ether for 4 cycles: 98%, 98%, 98%, 98%).

1-8. (canceled)

9. A method for dispensing a fluorous reaction component comprising:
dispensing by a non-gravimetric method a desired amount of the fluorous reaction component, wherein the fluorous reaction component is a coating on at least a fluorous support material; and
adding the fluorous support material coated with the desired amount of the fluorous reaction component to a reaction vessel.

10. The method of claim 9, wherein dispensing by the non-gravimetric method comprises one of measuring a unit length of the fluorous support material, measuring a unit area of the fluorous support material, measuring a volume of fluorous support material, and counting out a number of the fluorous support material.

11. The method of claim 9, wherein the fluorous support material is selected from the group consisting of polytetrafluoroethylene tape, fluoropolymer tape, fluoropolymer sheets, fluoropolymer mesh, fluoropolymer rods, fluoropolymer bars, a fluoropolymer reaction vessel, a fluoropolymer stir blade, a fluoropolymer stir bar, a fluoropolymer plug, a fluoropolymer liner, and fluoroplymer fluoropolymer beads.

12. The method of claim 11, wherein the fluorous support material is polytetrafluoroethylene tape and dispensing by the non-gravimetric method a desired amount of the fluorous reaction component comprises cutting a measured length of the polytetrafluoroethylene tape.

13. The method of claim 11, wherein the fluorous support material is a fluoropolymer bead, and dispensing by the non-gravimetric method a desired amount of the fluorous reaction component comprises one of measuring a volume of fluorous support material, and counting out a number of the fluorous support material.

14. The method of claim 9, wherein the fluorous reaction component is one of a fluorous reagent and a fluorous catalyst, the fluorous reaction component having a formula:
D[(R)ₙ(Rf)ₘ]_{y},
wherein D has a structure selected from the group consisting of an organic group, P, OH, OR, N, S, As, and Si; R is independently, the same or different, a hydrocarbon moiety; Rf is independently, the same or different, a fluorous moiety, n is an integer greater than or equal to 0; m is an integer greater than 0; and y is an integer from 1 to the maximum number of bonding attachments of D.

15. The method of claim 9, wherein the fluorous reaction component is one of a fluorous reagent and a fluorous catalyst, the fluorous reaction component having a formula:
Mₓ{L[(R)ₙ(Rf)ₘ]_{y}}_{z},
wherein M is a metal selected from the group consisting of a transition metal, a lanthanide metal, thorium, uranium, and a main-group metal; L is a ligand core having a structure selected from the group consisting of C, N, O, P, As, S, and Si; R is independently, the same or different, a hydrocarbon moiety; Rf is independently, the same or different, a fluorous moiety, n is an integer greater than or equal to 0; m is an integer greater than 0; y is an integer from 1 to the maximum number of bonding attachments of L; z is an integer from 1 to the maximum number of ligands attachable to M; and x is an integer from 1 to 4.

16. A method for forming a fluorous delivery and recovery material comprising:
depositing a fluorous reaction component as a coating on at least a portion of a surface of a fluorous support material, wherein the fluorous support material is capable of being applied to a reaction by a non-gravimetric method.

17. The method of claim 16, the method further comprising:
dissolving an amount of the fluorous reaction component in a solvent;
adding the fluorous support material to the solution; and
evaporating the solvent, whereby the fluorous reaction component is deposited as a coating on at least a surface of the fluorous support material.

18. The method of claim 16, wherein the fluorous support material is polytetrafluoroethylene tape, wherein the fluorous support material is capable of being applied to a reaction by cutting a measured length of the fluorous support material.

19. The method of claim 16, wherein the fluorous reaction component is one of a fluorous reagent and a fluorous catalyst, the fluorous reaction component having a formula:
D[(R)ₙ(Rf)ₘ]_{y},
wherein D has a structure selected from the group consisting of an organic group, P, OH, OR, N, S, As, and Si; R is independently, the same or different, a hydrocarbon moiety; Rf is independently, the same or different, a fluorous moiety, n is an integer greater than or equal to 0; m is an integer greater than 0; and y is an integer from 1 to the maximum number of bonding attachments of D.

20. The method of claim 16, wherein the fluorous reaction component is one of a fluorous reagent and a fluorous catalyst, the fluorous reaction component having a formula:
Mₓ{L[(R)ₙ(Rf)ₘ]_{y}}_{z},
wherein M is a metal selected from the group consisting of a transition metal, a lanthanide metal, thorium, uranium, and a main-group metal; L is a ligand core having a structure selected from the group consisting of C, N, O, P, As, S, and Si; R is independently, the same or different, a hydrocarbon moiety; Rf is independently, the same or different, a fluorous moiety, n is an integer greater than or equal to 0; m is an integer greater than 0; y is an integer from 1 to the maximum number of bonding attachments of L; z is an integer from 1 to the maximum number of ligands attachable to M; and x is an integer from 1 to 4.
